(19) United States
(12) Patent Application Publication
(10) Pub. No.: US 2013/0252534 A1
(43) Pub. Date: Sep. 26, 2013

(54) WASTE RECEPTACLE WITH IMPROVED VENTING SYSTEM AND DEODORIZING SYSTEM

(71) Applicant: John G. Smith, Louisville, KY (US)

(72) Inventor: John G. Smith, Louisville, KY (US)

(21) Appl. No.: 13/427,517

(22) Filed: Mar. 22, 2012

(51) Int. Cl. F24F 7/00; A61L 9/00

(52) U.S. Cl. 454/237; 422/243

(57) ABSTRACT

The present invention relates to a deodorizing system for a waste receptacle with a venting system. More particularly, the present invention relates to a waste receptacle that contains a venting system which allows air to enter the receptacle when removing a trash liner, thus allowing the liner to be removed more easily than before. Additionally, as the air moves through the venting system and across or through a deodorizing media, a fresh scent, anti-bacterial agent or other desired effect is provided when the garbage liner is removed from the waste receptacle.

### CROSS-REFERENCE TO RELATED APPLICATION

This application claims the benefit of U.S. Provisional Patent Application No. 61/192,419, filed Sep. 18, 2008, and U.S. Ser. No. 12/586,206, filed Sep. 18, 2009, entitled WASTE RECEPTACLE WITH IMPROVED VENTING SYSTEM AND DEODORIZING SYSTEM, both of which are incorporated by reference herein.

### BACKGROUND OF THE INVENTION

(a) Field of the Invention

The present invention relates to waste receptacles and other containers or objects that receive and support liners, such as trash bags. More particularly, the invention relates to a pressure differential relief waste receptacle that is arranged to prevent pressure differential adherence of a waste receptacle liner to the waste receptacle during removal of the liner.

(b) Background of the Invention

Trash receptacles are a common household item often found in several areas of the home. Trash liners are inserted in trash receptacles and are typically plastic and conform and adhere to the inside of the receptacle as objects are received therein. When trash liners are removed from the receptacle, the liner and its contents often weigh several pounds, and require the person removing the liner to be able to lift the weight of the liner and its contents above the top of the receptacle using a substantially vertical motion. In addition to the weight of the liner and its contents, the act of removing the liner from the receptacle is made even more difficult because as the liner adheres to the inside surface a pressure differential occurs in the receptacle. This pressure differential acts on the liner removal making it more difficult and physically demanding. The term vacuum as used herein is not intended to refer to an absolute absence of air or matter but rather to an enclosed space from which matter, especially air, has been partially removed or simply does not occupy so that the matter or gas occupying the space exerts less pressure than the ambient atmosphere or immediately surrounding condition. Vacuum is used broadly to refer to a pressure differential between the inside of a receptacle and the outside of the receptacle that is created when removing a liner in the absence of a flow path for air to enter and fill the expanding void/area defined by the receptacle interior and the exterior of the liner. This pressure differential requires that the person removing the liner act with an equal and opposite force in order to remove the liner. This task is often difficult for and can cause injury to the elderly, the infirm, and people with a low center of gravity, such as children. Moreover, in commercial and industrial settings much larger receptacles are used to hold even greater amounts of refuse having even greater weight. This greater weight when combined with the vacuum effect can result in an excessive physical demand and can lead to injury to even robust individuals.

An additional problem long associated with trash receptacles is that undesirable odors, bacteria and other problems develop over time. Cleaning is often the only way to deal with this problem and that is on a periodic basis. What is needed is a way to continually address the problems enumerated above to provide a more pleasant experience when dealing with and maintaining trash receptacles.

While there are known receptacles that include a venting means to help relieve the problem of a pressure differential when removing trash liners, what is needed is a system that addresses all of these problems and does so in an efficient and trouble-free manner.

### SUMMARY OF THE INVENTION

The present invention provides a combined deodorizing and venting system for waste receptacles. A venting or vacuum relief system is included to decrease the pressure differential created when a trash liner is removed from a waste receptacle. When the liner is removed from a receptacle with a venting system, the pressure differential between the outside of the receptacle and the inside of the receptacle causes air to move from outside the receptacle, through the intake opening, down the air conduit, and out the exhaust opening of the venting system. This air flow avoids a pressure differential that would otherwise occur, thus making it easier to remove the liner from the receptacle. The present invention modifies this process so that the air that is moving into the receptacle is acted on by a deodorizing media so that the air entering the receptacle is conditioned by the deodorizing media prior to entering the receptacle. This media can be designed to freshen the air, deodorize or sanitize the air, kill bacteria in the receptacle, prevent germs, mold, smells, fungus, insects, and/or other undesired conditions, or any combination thereof. However, the present invention may be used in many environments and the invention is not limited to the specific applications discussed herein.

While there are known trash can vent systems, including the Brawny Ultimate Trash System™, none of the vent systems include a deodorizing feature. This feature is needed to combat the odor that builds up as garbage sits in the receptacle. Odor, mold, bacteria and under undesirable conditions often build up as the waste receptacle is used over time, and can be especially troublesome in outdoor waste receptacles or large commercial or industrial receptacles where the garbage sits for periods of time. Odors draw bugs and microbes and often linger after the garbage has been removed and a new liner has been placed in the container. Also, in homes when receptacles are kept in cabinets or closets, the odors can build up causing the entire area to smell like garbage. This is especially true in the kitchen. In particular, restaurants often place waste receptacles with liners within enclosures such as within lobby areas where patrons discard food and other waste after dining via swinging panels or openings. The present invention provides an improved dining experience by preventing the waste enclosure from exhibiting the previously discussed undesirable and potentially unhealthy conditions. This deodorizing feature works in conjunction with the venting system so that as the liner is removed and air is pulled through the air conduit in response to the pressure differential, the vent air is pulled across or through a deodorizing media, causing the air to be freshened and otherwise conditioned as the liner is removed. The deodorizing media can be a product designed to deodorize, to freshen the air, or an antibacterial media or a combination thereof.

In one embodiment, the deodorizing media is placed in the air conduit at the intake opening, located at the top of the waste receptacle. The media is held in place by a shelf, ridge, shoulder, or other support located in the air conduit, and the deodorizing media can be attached to a hook that extends over the lip of the waste receptacle to hold it in place.

In another embodiment, the air conduit has a hinged opening on the outer wall of the waste receptacle. This allows the deodorizing media to be placed at the hinged opening. The hinged opening may be at the bottom of the outer wall of the waste receptacle, or at any other point on the waste receptacle. If the hinged opening is not at the bottom of the outer wall, a shelf must be present in the air conduit just under the hinged opening to allow the deodorizing media to rest on the shelf for easy replacement.

In another embodiment, the waste receptacle does not contain air conduits, but rather a vent in the wall or bottom of the receptacle. Around the vent on the inside of the receptacle is a vented enclosure that holds the deodorizing media, allowing air to enter the enclosure through the vent in the receptacle wall, pass across or through the deodorizing media, and into the receptacle.

In another embodiment, the invention provides a trash receptacle comprising: at least one sidewall; a bottom having a perimeter at which the bottom adjoins at least one sidewall to form an open top for receiving a removable trash liner and to form a partially enclosed area for supporting the removable trash liner, the partially enclosed area including a lower interior portion; means for providing a vacuum relief, whereby upon removal of the trash liner ambient air is introduced into the lower interior portion to facilitate removal of the trash liner from the enclosed area; and means for receiving and supporting a deodorizing agent, whereby the ambient air introduced into the lower interior portion through the vacuum relief means is acted upon by the conditioning deodorizing agent prior to being introduced into the lower interior portion.

In another embodiment, the invention provides a cartridge for use in a vented trash receptacle, the cartridge comprising: an outer surface at least in part configured to be received into a vacuum relief component of a trash receptacle; an intake adapted to receive ambient air drawn into the vacuum relief component of the receptacle; an output adapted to output the ambient air into the vacuum relief component for delivery to an area within the receptacle of reduced air pressure when compared to the ambient air pressure; and an agent adapted to condition the ambient air received at the intake, the conditioned ambient air being delivered at the output for delivery to the area of reduced air pressure to produce a desired effect.

### BRIEF DESCRIPTION OF THE DRAWINGS

A better understanding of the present invention will be had upon reference to the following description in conjunction with the accompanying drawings. These drawings should not be construed as limiting the present invention, but are intended to be exemplary and for reference, wherein:

FIG. 1 shows a cross-section of a trash receptacle incorporating a first embodiment of the present invention from the front view;

FIG. 2 shows a top down view of the embodiment of FIG. 1;

FIG. 3 shows an alternate top down view of the embodiment of FIG. 1;

FIG. 4 shows a cross-section of a trash receptacle incorporating a second embodiment of the present invention from the front view;

FIG. 5 shows a top down view of the second embodiment of FIG. 4;

FIG. 6 shows a cross-section of the third embodiment of a trash receptacle incorporating the present invention from the front view;

FIG. 7 shows a cross-section of the fourth embodiment of the present invention from the front view;

FIG. 8 shows a cross-section of the fifth embodiment of the present invention from the front view;

FIG. 9 shows a plurality of waste receptacles as shown in FIG. 4 arranged in a stacking fashion;

FIG. 10 shows a top down perspective of a first embodiment of the deodorizing media;

FIG. 11 shows a top down perspective of a second embodiment of the deodorizing media;

FIG. 12 shows a cross-section of a third embodiment of the deodorizing media disposed in the air conduit and having a support clip.

### DETAILED DESCRIPTION OF THE PREFERRED EMBODIMENT

The present invention will now be described in more detail with reference to exemplary embodiments as shown in the accompanying drawings. While the present invention is described herein with reference to the exemplary embodiments, it should be understood that the present invention is not limited to such exemplary embodiments. Those possessing ordinary skill in the art and having access to the teachings herein will recognize additional implementations, modifications, and embodiments, as well as other applications for use of the invention, which are fully contemplated herein as within the scope of the present invention as disclosed and claimed herein, and with respect to which the present invention could be of significant utility.

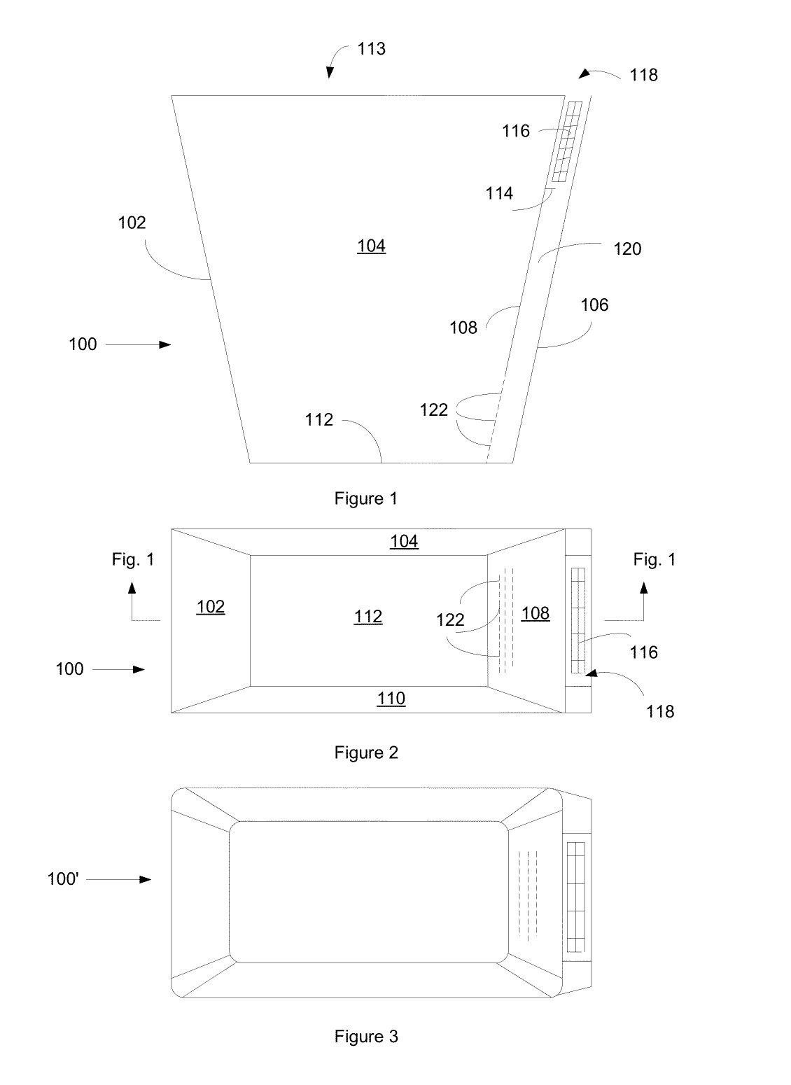

Referring now to FIGS. 1 and 2, a waste receptacle 100 is shown comprised of sidewalls 102, 104, 106, and 110, interior wall 108, and bottom 112 that joins the sidewalls so as to form an opening 113 at the top of the receptacle. An air conduit 120 is formed between inner sidewall 108 and outer sidewall 106 and in the configuration as shown extends along the height of the waste receptacle, with an intake opening 118 at the top of the waste receptacle and a plurality of exhaust openings 122 at the bottom of the waste receptacle. It should be understood that the number and size of openings at the intake and at the exhaust is a design choice and is not critical to the invention. However, the intake opening(s) conduit dimensions, and exhaust opening(s) are configured to allow effective flow of relief air into the receptacle to compensate for the pressure differential accruing therein during trash liner removal.

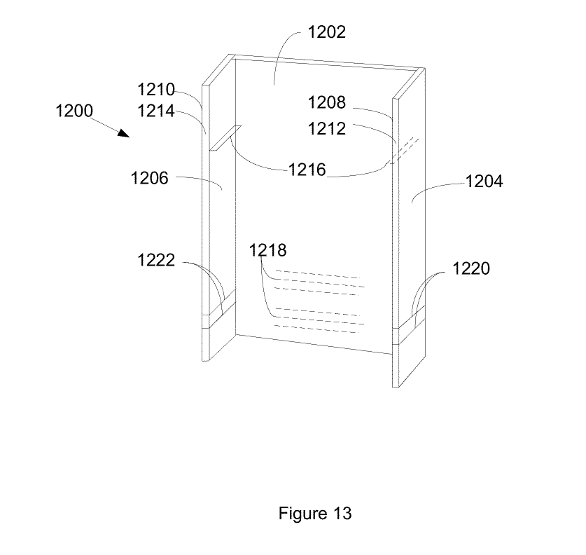

Near the intake opening 118, at or near the top portion of the air conduit 120, there is a shelf, ridge, tab, shoulder, or other suitable support 114 that extends from the inner sidewall 108 into the space of the air conduit 120 and provides a support for deodorizing media cartridge 116. This shelf 114 can take any form adapted for the purpose of supporting the cartridge 116 and may be configured to cooperate with the shape of the cartridge and the dimensions of the conduit 120 and may also extend from sidewall 106 with no material change to the invention. When inserted into vacuum relief conduit 120, the deodorizing media 116 rests on top of or upon shelf 114. In the alternative, the cartridge may be wedge or frusto-conical in shape, for instance, to cooperate with the shape of the conduit, or at least that portion of the conduit that receives the cartridge. In this manner the cartridge 116 may be supported by the wall structure of the receptacle without the need for a shelf. Also, the media cartridge may include a tab, handle, or other means to allow a person to grab and remove the cartridge from within the conduit for replacement or for recharging the cartridge with active material, such as disinfectant or deodorizing agent. Further the deodorizing media 116 may be held in place by a hanger, clip or other suitable component such as by extending over the sidewall of the waste receptacle as shown in FIG. 12 to hang in place.

FIG. 2 shows a top down view of the receptacle with intake opening 118 of vacuum relief air conduit 120 and deodorizing media cartridge 116 situated between inner sidewall 108 and outer sidewall 106. At the bottom of the inner sidewall 108 are exhaust openings 122 for the air conduit. In operation, when a liner, such as a plastic trash bag, is placed within receptacle 100 it conforms to the shape of the receptacle and comes in contact with the sidewalls and bottom. Upon receiving disposed of items, plastic liners typically adhere or cling to the interior sidewalls and bottom of the receptacle. When removing the liner from the receptacle, a pressure differential occurs between the space at the interior bottom of the receptacle and the ambient surrounding thereby causing ambient air to be drawn into the conduit 120 through opening 118 and across deodorizing media 116 thereby conditioning the ambient air with the substance(s) included within the deodorizing media cartridge 116. While described in terms of deodorizing media, the invention does not require that odor be removed by the media but rather a desirable scent or other effect may be applied. As used herein, the term “conditioning agent” shall be used to refer to deodorizing agents, disinfectants, anti-mold, anti-fungal, insect repellant, perfume, anti-bacterial, and other products as desired and as may be suggested by the particular application of the invention, i.e., depending on the environment and use of the receptacle. The conditioned ambient air is then introduced into the expanding volume in the interior of the receptacle as the liner is removed via exhaust openings 122, which may take any shape, such as slits, holes, gaps, etc. Also, the opening(s) may be located anywhere along the sidewall, but is/are preferable at or near the bottom of the receptacle. Multiple openings 122 may be located along the sidewall. Likewise, intake opening 118 does not have to be located at the very top of the receptacle. The key is to provide an opening so that ambient air may be introduced into the receptacle. Other exemplary arrangements are discussed hereinbelow.

Once the liner is removed, the person removing the liner preferably will notice a pleasing scent emanating from the receptacle as a result of the operation of conditioning the ambient air delivered into the receptacle via the venting system, as opposed to a foul odor that otherwise might be present. Even if there is no foul odor, there is benefit to having a pleasing scent associated with removal of trash liners or in having a disinfectant, anti-bacterial, insect repellant, or other agent introduced into the trash receptacle. In this manner the invention accomplishes two or more goals simultaneously: it relieves the pressure differential and reduces the effort required to remove the liner, and it conditions the air introduced into the receptacle by way of the deodorizing/conditioning media cartridge.

While the invention is illustrated in these examples as being incorporated on one side of the receptacle, it is understood that the invention may be on two or more sides or locations and that multiple conduits may be used to promote even greater vacuum relief and/or greater conditioning of ambient air. Likewise, the invention may be incorporated into the corners of the receptacle. It is understood that manufacturing concerns may influence the particular configuration of the openings and conduit and deodorizing media. The particular configuration is not critical to the invention. Also, while the deodorizing media is described as being carried in a cartridge, it is understood that deodorizing agents may be introduced directly into the conduit or at the openings, such as by a spray bottle, liquid gel, or other type applicator. Also, a removable pan or holder for liquid, gel, or loose deodorizing agent(s) may be used in place of the cartridge or may be used to “charge” and “re-charge” cartridges over time. The key is to provide a means to condition the ambient air being introduced into the receptacle when the liner is being removed and the pressure differential relieved.

FIG. 3 shows a top down view of an alternative embodiment wherein the receptacle is configured with rounded corners. This embodiment shows a waste receptacle 100′ which is the same as the waste receptacle 100 in FIG. 2, but the corners of waste receptacle 100′ are rounded whereas the corners of waste receptacle 100 are sharper. It is appreciated that receptacles are often configured to accommodate and facilitate shipping of the products such as by making them stackable for economy of space and packaging. Preferably, the receptacle is designed to incorporate the present invention in a low profile manner to promote stackability of a plurality of receptacles for storing and shipping.

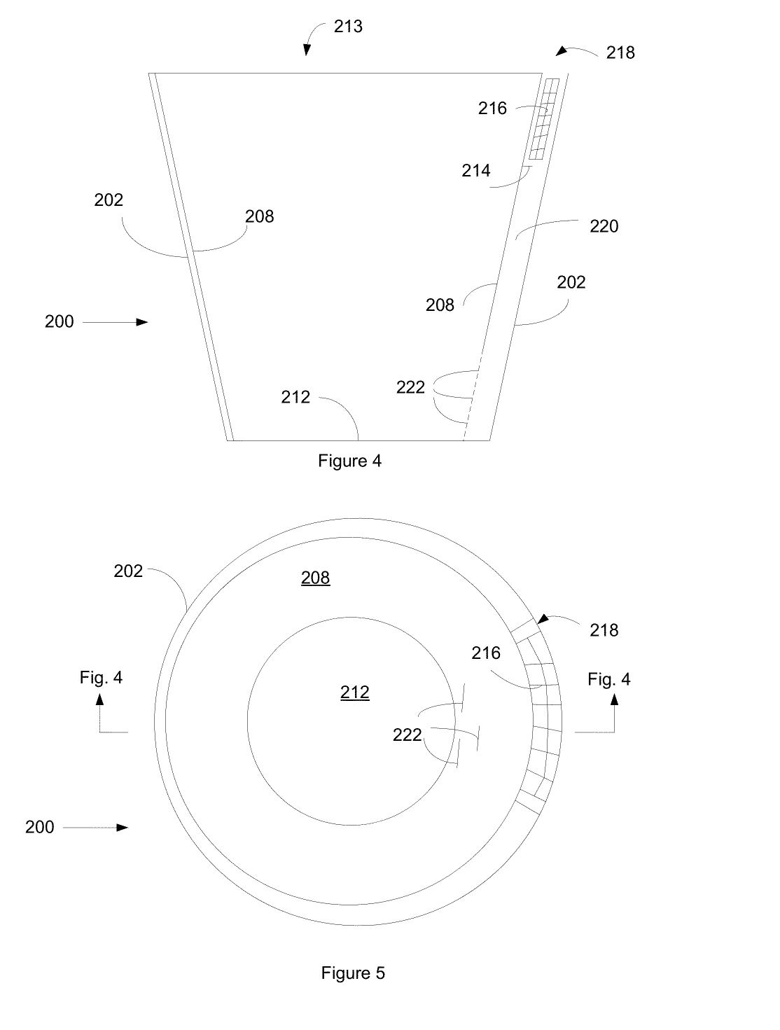

FIG. 4 shows a front cross-section of a second embodiment of the present invention. This embodiment shows a slightly tapered cylindrical waste receptacle 200 with a sidewall 202, a bottom 212, which is connected to the side wall 202, and an open top 213. The cylindrical waste receptacle 200 can be either double walled, or single walled. If the receptacle 200 is double walled, there is an inner sidewall 208. If receptacle 200 is single walled, 208 refers to the inside of the outer sidewall. There is an air conduit 220, which runs the height of the waste receptacle 200. At the top of the air conduit 220, there is an intake opening 218, and at the bottom of the air conduit there is/are exhaust opening(s) 222 into the waste receptacle 200. Near the top of the conduit 200, there is a shelf 214 that extends part of the way into the air conduit, and which supports the deodorizing media 216 while in the air conduit. The operation of the receptacle is as described above in the context of FIG. 1 and multiple conduits and openings may be incorporated in the receptacle. Like the embodiment of FIG. 1, the particular configuration of receptacle 200 is not critical to operation of the invention and manufacturing, storing, and shipping considerations may affect the design.

FIG. 5 is a top down view of waste receptacle 200. The intake opening 218 to the air conduit 220 is shown with the deodorizing media 216 in place. The exhaust openings 222 are shown as slits near the bottom of the waste receptacle. As mentioned above the opening(s) may take any shape and size.

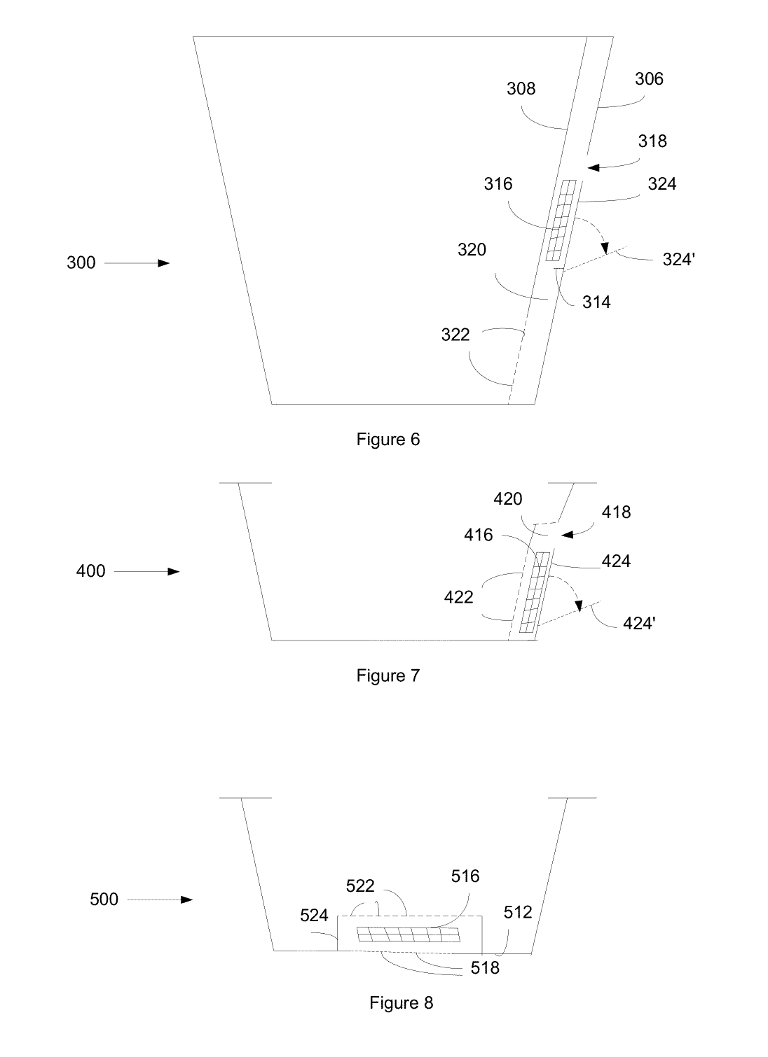

FIG. 6 shows a front cross-section of a third embodiment of the present invention. This embodiment shows a slightly tapered cylindrical waste receptacle 300 with an outer sidewall 306, and an inner sidewall 308, which form the air conduit 320. The intake opening 318 of the air conduit 320 is located near the middle of the waste receptacle 300. Just below the intake opening 318 on the outer sidewall 306 of the waste receptacle 300 is a hinged flap 324. The hinged flap 324 has a closed position 324 and an open position 324′. When the hinged flap is in the open position 324′ the deodorizing media 316 is placed in the air conduit and rests on the shelf 314 which extends from the outer sidewall into the air conduit. The flap may include a ledge or shelf to support the cartridge or may include a pocket or other means for receiving loose deodorizing agents. Near the bottom of the air conduit 320, are the exhaust openings 322.

FIG. 7 shows a lower portion of an alternative configuration in cross-section of a waste receptacle 400. This fourth embodiment shows an air conduit 420 which has an intake opening 418 near the bottom of the receptacle 400. Immediately below the intake opening 418 on the outer sidewall of the waste receptacle 400 is a hinged flap 424. The hinged flap 424 has a closed position 424 and an open position 424′. When the hinged flap is in the open position 424′ the deodorizing media 416 is placed in the air conduit and rests on the bottom of the air conduit 420, which is at the bottom of the sidewall of the receptacle 400. Opposite the hinged flap 424, are the exhaust openings 422.

FIG. 8 shows yet another alternative embodiment of a receptacle 500 having a compartment 524 for receiving deodorizing media 516 and having intake opening(s) 518 located on the bottom wall 512 of the receptacle and exhaust opening(s) 522 located on the top of compartment 524. The compartment 524 may extend across the to the sides of the receptacle to provide a flush or flat surface at the bottom of the interior of the receptacle. In this configuration, the intake openings may be located on the sides of the receptacle wall(s). Again, the key is not the particular configuration but to bring in ambient air, to condition the ambient air, and to introduce the conditioned ambient air into the lower portion of the receptacle when removing a trash liner.

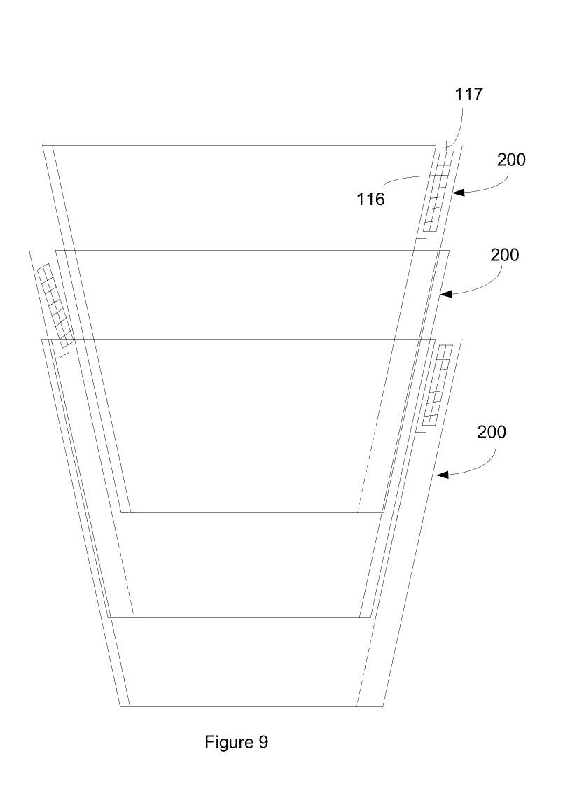

FIG. 9 shows a plurality of waste receptacles 200 stacked in a alternating configuration. By alternating the side the air conduit is located, the waste receptacles 200 can be stacked without leaning to one side. Preferably, the receptacle incorporating the invention is configured in a way that promotes stackability and minimizes space for storing and shipping. As shown in FIG. 9, conditioning cartridge 116 may contain a pull-tab 117 for facilitating inserting and removing conditioning cartridge 116.

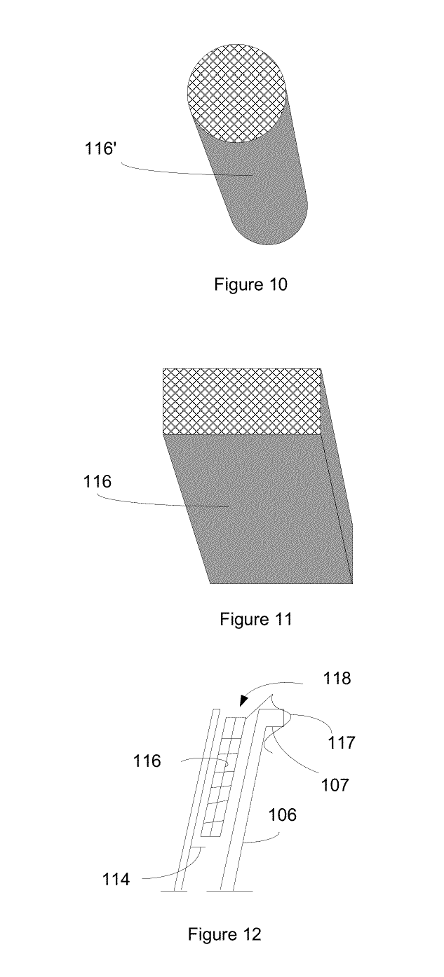

FIG. 10 shows a cylindrical-shaped deodorizing media 116. The media 116 may take on different shapes and materials depending on manufacturing costs as well as effectiveness. The deodorizing media 116 could be liquid-based, paper-based, gel-based, carbon filter type, or aerosol, for example.

FIG. 11 shows a rectangular-shaped deodorizing media 116′. The media 116′ may take on different shapes and materials depending on manufacturing costs as well as effectiveness. The deodorizing media 116′ could be liquid-based, paper-based, gel-based, carbon filter type, or aerosol. Again, although the invention is described in terms of deodorizing media, any one or combination of desired agents may be used, e.g., perfume, scents, anti-bacterial substances, anti-fungal substances, mold retardant, insecticides, charcoal-based substances, and such agents may take solid, liquid or gas form.

FIG. 12 shows the apparatus 117 for holding the deodorizing media 116 in a removeably fixed manner in the air conduit. The apparatus 117 hooks over the lip 107 of the waste receptacle at the intake opening 118 of the air conduit. The shelf 114 that extends part way into the air conduit is optional and can be used to help support the deodorizing media 116 and may be used in conjunction with the apparatus 117 or separately. Tape or other adhesive, Velcro, or simply the shape of the deodorizing cartridge may operate to hold it in place when positioned in the conduit.

In one embodiment, the invention provides a trash receptacle comprising: at least one sidewall; a bottom having a perimeter at which the bottom adjoins at least one sidewall to form an open top for receiving a removable trash liner and to form a partially enclosed area for supporting the removable trash liner, the partially enclosed area including a lower interior portion; means for providing a vacuum relief, whereby upon removal of the trash liner ambient air is introduced into the lower interior portion to facilitate removal of the trash liner from the enclosed area; and means for receiving and supporting a deodorizing agent, whereby the ambient air introduced into the lower interior portion through the vacuum relief means is acted upon by the conditioning deodorizing agent prior to being introduced into the lower interior portion.

In another embodiment, the invention provides a cartridge for use in a vented trash receptacle, the cartridge comprising: an outer surface at least in part configured to be received into a vacuum relief component of a trash receptacle; an intake adapted to receive ambient air drawn into the vacuum relief component of the receptacle; an output adapted to output the ambient air into the vacuum relief component for delivery to an area within the receptacle of reduced air pressure when compared to the ambient air pressure; and an agent adapted to condition the ambient air received at the intake, the conditioned ambient air being delivered at the output for delivery to the area of reduced air pressure to produce a desired effect.

With reference to FIG. 13, an alternative embodiment of the present invention provides a device for attaching to an existing waste receptacle, such as in a retro-fit application or as a process in the manufacture of a new-for-sale trash can. A vent system 1200 includes a wall 1202 connected at either side to sidewalls 1204 and 1206, which respectively have ends 1208 and 1210. In one embodiment, adhesive strips 1212 and 1214 may respectively be located along the lengths, or along parts of the lengths, of ends 1208 and 1210. In addition, adhesive strips may be located along the bottom edges of one or more of walls 1202-1206 to secure the system at the bottom of the receptacle. One or more vent system(s) 1200 may be fastened to the interior surface of a waste receptacle, e.g., along the interior surface of wall 102 or 104 or 108 or 110 of receptacle 100 of FIG. 1, by removing a temporary strip and exposing the adhesive and applying to the surface of the receptacle wall. The invention is not limited to this form of fastening the system 1200 to the receptacle. For instance, the receptacle may be formed with a cooperative protrusion(s), tab(s), hole(s), channel(s), etc., to receive a counter-formed protrusion(s), tab(s), hole(s), channel(s), etc., formed along the edges 1208, 1210 of the system 1200 to effect fastening of the system 1200 to the receptacle. In another embodiment (not shown), the vent system 1200 may be enclosed on all sides along the length of the vent system 1200 such that the adhesive strips or other fastening means are on the exterior of the vent system 1200.

The vent system should be oriented so that vent slits, holes or openings 1218, which may be formed in one, some or all of walls 1202-1206, are located at or near the bottom portion of the receptacle and the opposite end of the system 1200 is located near the top of the receptacle when installed. The walls 1202, 1204, 1206 when attached to the interior of a trash receptacle form an open-ended conduit through which air may enter at the open top end and pass through the conduit and exit exhaust vents 1218 at the bottom to relieve any pressure differential that occurs when removing trash liner from the receptacle. In addition, the vent system may be installed to create a gap at the bottom to allow air to pass through to the bottom. This may be in addition to openings 1218 or in place of openings 1218, i.e., no openings need be formed at all. In addition, the walls 1202-1206 may be of different lengths to create one or more gaps. For instance, the wall 1202 may be longer than walls 1204-1206, whereby a gap is formed on both sides of the conduit when the system is installed with the bottom edge of wall 1202 flush with the upper surface of the bottom of the receptacle. Thus the vent system 1200 operates as described above for other embodiments. Also as described above in relation to the conditioning of the vent air that enters into the bottom space of the receptacle, shoulders, shelves or protrusions 1216 are provided to support a conditioning cartridge in place in the conduit upon installation of the vent system. In the alternative, the vent system may be tapered to narrow from top to bottom so as to “trap” the cartridge and hold it in place by supporting the cartridge along the inner surface of walls 1204 and 1206. Also, a tab, handle, protrusion or other means for grasping the cartridge may be provided to facilitate removal of the cartridge for replacement or recharging the device with conditioning media, such as deodorizing agent, disinfectant, perfume or scented agents, etc.

Vent system 1200 is shown in rectangular form but may be configured in any desired shape, e.g., curved, to achieve compatible fit and accurate installation with a variety of receptacle shapes. Optional indentions, creases, slits or other suitable means 1220 may be provided in one or more of walls 1202-1206 to enable a person to alter the length of the walls, and thus the length of the vent system 1200, to accommodate fitting and installation of the device to a variety of sizes of receptacles.

The present invention is not to be limited in scope by the specific embodiments described herein, It is fully contemplated that other various embodiments of and modifications to the present invention, in addition to those described herein, will become apparent to those of ordinary skill in the art from the foregoing description and accompanying drawings. Thus, such other embodiments and modifications are intended to fall within the scope of the following appended claims. Further, although the present invention has been described herein in the context of particular embodiments and implementations and applications and in particular environments, those of ordinary skill in the art will appreciate that its usefulness is not limited thereto and that the present invention can be beneficially applied in any number of ways and environments for any number of purposes.

What is claimed is:

1. A cartridge for use in a vented trash receptacle, the cartridge comprising:
an outer surface at least in part configured to be received into a pressure differential relief component of a trash receptacle;
an intake adapted to receive ambient air drawn into the pressure differential relief component of the receptacle;
an output adapted to output the ambient air into the pressure differential relief component for delivery to an area within the receptacle of reduced air pressure when compared to the ambient air pressure; and
an agent adapted to condition the ambient air received at the intake, the conditioned ambient air being delivered at the output for delivery to the area of reduced air pressure to produce a desired effect.

2. The cartridge of claim 1, wherein the cartridge is configured such that the agent contained in the cartridge may be recharged.

3. The cartridge of claim 2, wherein recharging includes at least one of the following: removing old agent from the cartridge and adding new agent to the cartridge.

4. The cartridge of claim 1, further comprising a tab for inserting the cartridge into and extricating the cartridge from the vented trash receptacle.

5. A vent system for mounting inside a trash receptacle, comprising:
an elongated channel having a top end and a bottom end and being open at the top end;
means for adhering the elongated channel to the trash receptacle such that when the elongated channel is joined to the trash receptacle, an air passageway defined at least in part by the elongated channel permits air to enter and pass through the air passageway from the top end located near an open portion of the trash receptacle and to exit the passageway at or near the bottom end located near a bottom portion of the trash receptacle, the air providing a pressure differential relief to facilitate the removal of a trash liner from the trash receptacle;
means for removeably receiving and supporting a conditioning agent, whereby the air passing through the passageway is acted upon by the conditioning agent.

6. The vent system of claim 5, wherein the elongated channel is joined to the trash receptacle by one or more adhesive strips.

7. The vent system of claim 5, wherein the vent system is configured to be height-adjustable such that the vent system may fit trash receptacles of varying sizes and dimensions.
